(19) United States
(12) Patent Application Publication
(10) Pub. No.: US 2007/0026463 A1
(43) Pub. Date: Feb. 1, 2007

(54) Compositions and methods of mutant Nogo-66 domain proteins

(75) Inventors: Yuhong Xie, Chestnut Hill, MA (US); Brian Bates, Chelmsford, MA (US); Janet Paulsen, Londonderry, NH (US)

Correspondence Address: WILMER CUTLER PICKERING HALE AND DORR LLP /, 60 STATE STREET, BOSTON, MA, 02109, US

(73) Assignee: Wyeth, Madison, NJ (US)

(21) Appl. No.: 11/495,061

(22) Filed: Jul. 28, 2006

Related U.S. Application Data
(60) Provisional application No. 60/703,134, filed on Jul. 28, 2005.

(51) Int. Cl. C40B 30/06; G01N 33/53; C07H 21/04; C12P 21/06; C07K 14/705

(52) U.S. Cl. 435/7.1; 435/69.1; 435/320.1; 435/325; 530/350; 536/23.5

(57) ABSTRACT

To facilitate the study of Nogo-66 interaction with its neuronal receptors, and to explore therapeutic opportunities, the present invention provides mutant human Nogo-66 domain-based proteins that do not aggregate during isolation or purification procedures, and methods for using these proteins. Aggregates of Nogo-66 domain containing proteins do not effectively or efficiently bind to the NgR1 receptor, and therefore limit the utility of Nogo-66 domain-based reagents. To overcome aggregation problems, the invention provides proteins that comprise a mutant human Nogo-66 domain, wherein the cysteine at position 47 of the wild-type human Nogo-66 domain is mutated. The invention also provides for various Nogo-66 domain fusion reporter proteins that are able to bind to NgR1, and therefore can be used in high-throughput assays to identify drug candidate compounds that can block binding of the Nogo-66 domain to NgR1, indicating that these compounds may be potential therapeutic agents for neurodegenerative diseases and neuronal repair.

This application claims priority to U.S. Application No. 60/703,134 filed on Jul. 28, 2005, which is hereby incorporated by reference in its entirety.

### BACKGROUND OF THE INVENTION

All patent applications, published patent applications, issued and granted patents, texts, and literature references cited in this specification are hereby incorporated herein by reference in their entirety.

The inability of central nervous system (CNS) neurons to regenerate damaged axons severely limits patient recovery after traumatic injury, stroke, or certain neurodegenerative diseases. Regenerative failure of damaged axons has been attributed in part to proteins associated with CNS myelin and the scar that forms at an injury site. Multiple factors present in myelin, including the myelin inhibitory ligands, Nogo-A, myelin-associated glycoprotein (MAG) and oligodendrocyte-myelin glycoprotein (OMgp), have been demonstrated to be partly responsible for the impoverished neuronal repair response following CNS injury.

A common receptor, NgR1, has been identified to be capable of independently binding to the three myelin inhibitory ligands. NgR1 is a 473-residue GPI-linked, neuronal, axonally expressed protein with no intrinsic means of intracellular signaling. Signal transduction is mediated through an NgR1/p75 NTR/Lingo-1 trimeric co-receptor complex and with the cell by activation of Rho GTPases. Both p75 and Lingo-1 have been demonstrated to be required for signal transduction. Recently, another co-receptor, Troy (also known as Taj), has been demonstrated to form a functional receptor complex with NgR and Lingo-1 to mediate cellular responses to myelin inhibitors. Because NgR1 coordinates inhibition by interactions with multiple myelin associated inhibitory proteins, NgR1 represents a convergent target for drugs that can block myelin-derived inhibition of neuronal repair.

Nogo-66 is an axon-inhibiting domain located at the C-terminal end of the NogoA protein. The Nogo-66 domain consists of 66 amino acids that are displayed at the extracellular surface and at the endoplasmic reticulum lumen of transfected cells and oligondendrocytes. To facilitate the study of Nogo-66 interaction with its neuronal receptors, and to explore therapeutic opportunities, the present invention provides Nogo-66 domain-based fusion proteins that do not aggregate during isolation or purification procedures, and methods for using these fusion proteins.

### SUMMARY OF THE INVENTION

The invention relates to proteins and peptides that comprise a mutant human Nogo-66 domain. A key aspect of the present proteins and peptides lies in the introduction of a mutation into the coding sequence for Nogo-66. The mutation is at amino acid position 47 of the Nogo-66 peptide, where position 47 is cysteine in the wild-type sequence. Mutating position 47 from a cysteine to another amino acid, for example, valine, prevents Nogo-66 domain proteins from aggregating. Aggregation of wild-type Nogo-66 domain-containing proteins into high molecular weight complexes has limited the utility of Nogo-66 domain-based reagents. Aggregate complexes of wild-type Nogo-66-containing proteins hamper the ability to make quantitative assessments of Nogo-66/NgR1 interactions, and therefore limits the utility of Nogo-66 domain-based reagents. The present invention overcomes this aggregation problem, and provides for various proteins comprising a mutant Nogo-66 domain that can be used in assays based on the detection of NgR1 receptor/ligand interactions.

In one aspect, the invention provides a protein or peptide that comprises a mutant human Nogo-66 domain, wherein the mutant human Nogo-66 domain does not comprise a cysteine residue. In one aspect, the protein or peptide that comprises a mutant human Nogo-66 domain does not aggregate, wherein the mutant human Nogo-66 domain does not comprise a cysteine residue. In one aspect, the invention provides a mutant human Nogo-A protein or portion thereof comprising a Nogo-66 domain, wherein the Nogo-66 domain does not contain a cysteine residue, such that the mutant human Nogo-A protein does not aggregate. In one aspect, the invention provides a mutant human Nogo-66 protein or peptide that does not comprise a cysteine residue such that the protein or peptide does not aggregate.

The mutant human Nogo-66 domain proteins or peptides can be used alone or they can be used as domains in recombinant proteins. In one aspect, the invention provides a fusion protein, the fusion protein comprising a Nogo-66 domain that does not comprise a cysteine residue. The Nogo-66 domain can be fused to essentially any other peptide or protein, and mutating the cysteine residue at position 47 of the wild-type human Nogo-66 domain (see SEQ ID NO:3 for the wild-type sequence of the human Nogo-66 domain) allows for the fusion proteins to be isolated or purified in a non-aggregated form. The Nogo-66 domain can be at the N-terminus, C-terminus or in between the termini of the fusion protein.

The proteins and peptides of the invention can have a mutant human Nogo-66 domain comprising an amino acid sequence of SEQ ID NO:5, 6, 7, 8 or 9, or an amino acid sequence that is at least 95% or 99% identical to SEQ ID NO:5, 6, 7, 8 or 9 (provided that the cysteine residue is mutated). In one aspect, the invention provides a protein comprising a mutant human Nogo-66 domain consisting of or consisting essentially of an amino acid sequence of SEQ ID NO:5, 6, 7, 8 or 9. In one aspect, a protein or peptide of the invention comprises a mutant human Nogo-66 domain with an amino acid sequence of SEQ ID NO:5. In one aspect, the invention provides an antibody that is specific to a protein or peptide of the invention. In other words, a protein or peptide of the invention can be used as an immunogen to generate antibodies specific to the protein or peptide. In one aspect, the invention provides an antibody that is specific to an epitope of a protein or peptide of the invention, wherein the epitope comprises a mutation in the cysteine residue of the wild-type human Nogo-66 domain. In one aspect, the invention provides an antibody that has been raised against the mutant human Nogo-66 domain comprising an amino acid sequence of SEQ ID NO:5, 6, 7, 8 or 9. In another aspect, the antibody that is specific to a protein or peptide of the invention inhibits binding between a receptor and the wild-type or mutant human Nogo-66 domain. The receptor can be, for example, NgR1. Antibodies of the invention can be screened for their ability to inhibit binding between a receptor and the Nogo-66 domain according to the present methods for detecting whether a candidate molecule can inhibit binding between a Nogo-66 domain and a receptor.

In one aspect, the invention provides a fusion protein comprising: (a) a mutant human Nogo-66 domain that does not comprise a cysteine residue (including domains comprising the amino acid sequence of SEQ ID NO:5, 6, 7, 8 or 9); and (b) a reporter protein domain. In one aspect, the invention provides a non-aggregating fusion protein comprising: (a) a mutant human Nogo-66 domain that does not comprise a cysteine residue such that the fusion protein does not aggregate; and (b) a reporter protein domain.

For fusion proteins of the invention that comprise a reporter domain, the reporter protein domain can comprise, for example, an enzyme, a luminescent protein, or a fluorescent protein, or mutants, variants or portions thereof. In one aspect, the enzyme is the human placental secreted alkaline phosphatase protein or the β-galactosidase protein. In another aspect, the fluorescent or luminescent protein is a luciferase protein, a green fluorescent protein, a yellow fluorescent protein, a red fluorescent protein, or a blue fluorescent protein.

In one aspect, the fusion protein is secreted when expressed by a cell. The fusion protein can further comprise a leader sequence or a peptide signal sequence. The fusion protein can further comprise a cleavage site for an enzyme, such as a protease. In one aspect, the fusion protein further comprises an enterokinase cleavage site. In another aspect, the fusion protein further comprises an epitope tag. In one aspect, the epitope tag is a His₆ tag.

In one aspect, the invention provides a fusion protein comprising: (a) a mutant human Nogo-66 domain having the amino acid sequence of SEQ ID NO:5; and (b) an alkaline phosphatase domain.

In another aspect, the invention provides a fusion protein comprising the amino acid sequence of SEQ ID NO:16. In one aspect, the invention provides a fusion protein consisting essentially of the amino acid sequence of SEQ ID NO:16. The fusion protein having the amino acid sequence of SEQ ID NO:16 is both non-aggregating and is secreted when expressed by cells.

In one aspect, the invention provides a method for detecting whether a receptor can bind to a Nogo-66 domain (as used herein, a Nogo-66 domain includes Nogo-66 domain comprising proteins, such as Nogo-A), the method comprising: (a) contacting a receptor with a protein or peptide comprising a mutant human Nogo-66 domain, wherein the mutant human Nogo-66 domain does not comprise a cysteine residue; and (b) assaying for the presence of a protein complex comprising the receptor and the protein or peptide comprising a mutant human Nogo-66 domain. If a protein complex comprising the receptor and the protein/peptide comprising a mutant human Nogo-66 domain (i.e., the Nogo-66 domain does not comprise a cysteine residue such that protein/peptide does not aggregate) is detected, this indicates that the receptor can bind to the Nogo-66 domain. If the protein or peptide comprising a mutant human Nogo-66 domain also comprises a reporter domain, then the assaying step can involve assaying for reporter activity from the protein or peptide. Detection of reporter activity indicates that the receptor can bind to a Nogo-66 domain.

In one aspect, the invention provides a method for detecting whether a receptor can bind to a Nogo-66 domain, the method comprising: (a) contacting a receptor with a fusion protein of the invention comprising a reporter domain; and (b) assaying for reporter protein activity, wherein detection of reporter protein activity indicates that the receptor can bind to the Nogo-66 domain.

In the present methods, the receptor that is being contacted can be expressed on the surface of a cell. The receptor expressed by the cell can be encoded by a cDNA library expression vector that has been transfected into the cell. This allows high-throughput screening for receptors that are capable of binding to the Nogo-66 domain.

In one aspect, the invention provides a method for detecting whether a candidate molecule can inhibit binding between a Nogo-66 domain and a receptor, the method comprising: (a) contacting a receptor that binds to the Nogo-66 domain with: (i) a protein or peptide comprising a mutant human Nogo-66 domain, wherein the mutant human Nogo-66 domain does not comprise a cysteine residue, and (ii) a candidate molecule; and (b) assaying for the presence of a protein complex comprising the receptor and the protein or peptide comprising a mutant human Nogo-66 domain. If a protein complex of the receptor and the protein/peptide comprising a mutant human Nogo-66 domain (i.e., the Nogo-66 domain does not comprise a cysteine residue such that protein/peptide does not aggregate) is detected, or if lower amounts of the complex is detected as compared to samples where the candidate molecule was not added, this indicates that the receptor can bind to the Nogo-66 domain. The assaying for the presence of the protein complex can be conducted by standard biochemical techniques as described herein. If the protein or peptide comprising a mutant human Nogo-66 domain also comprises a reporter domain, then the assaying step can comprise assaying for fluorescence or luminescence. Detection of minimal amounts of fluorescence/luminescence or detection of lower amounts of fluorescence/luminescence as compared to control experiments where no candidate molecule is added, indicates that the candidate molecule can inhibit or otherwise negatively affect binding between the receptor and the Nogo-66 domain.

In another aspect, the invention provides a method for detecting whether a candidate molecule can inhibit binding between a Nogo-66 domain and a receptor, the method comprising: (a) incubating: (i) a receptor, (ii) a fusion protein of the invention comprising a reporter domain, and (iii) a candidate molecule; and (b) assaying for reporter protein activity, wherein no or minimal detection of reporter protein activity indicates that the candidate molecule can inhibit binding between Nogo-66 and the receptor. In this method, the receptor can be NgR1. Further, the method can be cell-based, where a cell expressing a receptor at its cell-surface is incubated. The fusion protein can have, for example, the amino acid sequence of SEQ ID NO:16. For the incubating step, the candidate molecule can be incubated with the cell prior to, at the same time, or after the addition of the fusion protein. Where the fusion protein comprises an alkaline phosphatase domain, the assaying step of the method can comprise adding a fluorescent or chemiluminescent substrate to the well (see Example 3 for a more detailed protocol). Further, assaying of reporter protein activity can involve determining whether there is a decrease of fluorescent or chemiluminescent emission from a sample having an addition of a candidate molecule as compared to a sample having no addition of the candidate molecule. In the invention, candidate molecules can also be described as drug candidates, and candidate molecules include, but are not limited to, chemical compounds like small-molecules, peptidomimetic chemical compounds that mimic the three-dimensional structure of the Nogo-66 domain, nucleic acids, proteins, peptides, and antibodies that bind to regions of the NgR1 receptor (including the region of the NgR1 receptor that binds to Nogo-66).

In the methods of the invention, the incubating or contacting or assaying can be conducted in individual wells of multi-well plates, including 96-well, 384-well and 1536-well microplates. These microplates can support cell-based assays, such that cells can be grown in culture media in these wells, therefore enabling direct assays on the cells. Further, the microplates are standardized to work with automation and detection equipment, such that methods that involve the detection of reporter activity can be conducted in a high-throughput manner.

In another aspect, the invention provides a method for testing whether a candidate molecule can inhibit binding between a Nogo-66 domain and a receptor, the method comprising: (a) incubating in a first well (or other container or substrate): (i) a cell expressing a receptor that binds to Nogo-66, (ii) a fusion protein of the invention, and (iii) a candidate molecule; (b) incubating in a second well: (i) a cell expressing the receptor that binds to Nogo-66, and (ii) the fusion protein used in step (a); and (c) assaying for reporter protein activity from the first and second well, wherein detection of decreased of reporter protein activity in the first well as compared to the second well indicates that the candidate molecule can inhibit the binding between the receptor and Nogo-66.

In one aspect, the invention provides a method for testing whether a candidate molecule can inhibit binding between Nogo-66 and NgR1, the method comprising: (a) incubating in a first well: (i) a cell expressing the NGR1 receptor on its cell surface, (ii) a fusion protein having the amino acid sequence of SEQ ID NO:16, and (iii) a candidate molecule; (b) incubating in a second well: (i) a cell expressing the NgR1 protein on its cell surface, and (ii) a fusion protein having the amino acid sequence of SEQ ID NO:16; and (c) assaying for reporter protein activity from the first and second well, wherein detection of decreased of reporter protein activity in the first well as compared to the second well indicates that the candidate molecule can inhibit the binding between the NgR1 receptor and Nogo-66. The assaying can comprise, for example, adding an alkaline phosphatase substrate to the first and second wells. As described in Example 3, if a candidate molecule (or “drug candidate”) can prevent binding of a fusion protein with NgR1, then the majority of the cells in a well will not have fusion proteins bound to their cell surface with NgR1—therefore, in such wells, there will be substantially little or no fusion proteins that can cleave the alkaline phosphatase substrates and thereby generate chemiluminescence or fluorescence.

### BRIEF DESCRIPTION OF THE FIGURES

FIG. 1 provides the amino acid sequence of the homo sapiens Nogo-A protein (SEQ ID NO: 1; Genbank Accession No. CAB99248). The Nogo-66 domain is located at amino acid residues 1055-1120 of SEQ ID NO:1.

FIG. 2 provides the amino acid sequence for an alternatively spliced form of homo sapiens Nogo-A (“short form”) (SEQ ID NO:2; Genbank Accession No. AAG40878), and the Nogo-66 domain is located at amino acid residues 823-888 of SEQ ID NO:2.

FIG. 3 provides the wild-type amino acid sequence of the human Nogo-66 domain (SEQ ID NO:3). The cysteine residue at position 47 is mutated in the fusion proteins of the invention in order to prevent Nogo-66 domain-based reagents from aggregating.

FIG. 4 provides the nucleotide sequence encoding the human Nogo-66 domain (SEQ ID NO:4). The nucleotides encoding the cysteine residue at position 47 of SEQ ID NO:3 is located at nucleotides 139-141 of SEQ ID NO:4. Nucleotides 139-141 can be mutated by standard procedures known to one skilled in the art, such as by site-directed mutagenesis.

FIG. 5 provides the amino acid sequence (SEQ ID NO:5) of a mutated human Nogo-66 domain used in various fusion proteins of the invention. The cysteine residue at position 47 of the wild-type Nogo-66 domain has been mutated to be a valine residue.

FIG. 6 provides the amino acid sequence (SEQ ID NO:6) of a mutated human Nogo-66 domain used in various fusion proteins of the invention. The cysteine residue at position 47 of the wild-type Nogo-66 domain has been mutated to be an alanine residue.

FIG. 7 provides the amino acid sequence (SEQ ID NO:7) of a mutated human Nogo-66 domain used in various fusion proteins of the invention. The cysteine residue at position 47 of the wild-type Nogo-66 domain has been mutated to be a glycine residue.

FIG. 8 provides the amino acid sequence (SEQ ID NO:8) of a mutated human Nogo-66 domain used in various fusion proteins of the invention. The cysteine residue at position 47 of the wild-type Nogo-66 domain has been mutated to be a leucine residue.

FIG. 9 provides the amino acid sequence (SEQ ID NO:9) of a mutated human Nogo-66 domain used in various fusion proteins of the invention. The cysteine residue at position 47 of the wild-type Nogo-66 domain has been mutated to be an isoleucine residue.

FIG. 10 provides a basic schematic of one possible fusion protein of the invention. The schematic shows that the fusion protein can comprise an alkaline phosphatase domain, an enterokinase cleavage site (EK), a mutated human Nogo-66 domain, and a 6-His tag (also referred to as “His₆”).

FIG. 11A-B provides the nucleotide sequence (SEQ ID NO:15) coding for a fusion protein (SEQ ID NO:16; FIG. 12) of the invention.

FIG. 12 provides the amino acid sequence (SEQ ID NO:16) encoded by the nucleotide sequence of SEQ ID NO:15. The amino acid sequence of SEQ ID NO:16 comprises the honey bee melatonin signal peptide (residues 1-21), a portion of the human placental secreted alkaline phosphatase protein (residues 28-516), an enterokinase protease cleavage site (residues 518-526), a human Nogo-66 domain having its cysteine mutated to a valine (residues 528-593, where the valine in place of a cysteine is at residue 574), and a His₆ epitope tag.

### DETAILED DESCRIPTION OF THE INVENTION

The invention provides proteins and peptides that comprise a mutant human Nogo-66 domain, where the cysteine residue in the Nogo-66 domain is mutated such that the proteins and peptides do not aggregate. Aggregate complexes of wild-type Nogo-66-containing proteins hamper the ability to make quantitative assessments of Nogo-66/NgR1 interactions, and therefore limits the utility of Nogo-66 domain-based reagents. The present invention overcomes this aggregation problem, and provides for various mutant Nogo-66 domain-comprising proteins. Because these mutant Nogo-66 domain-comprising proteins do not aggregate, and because they maintain their ability to bind to receptors that bind to Nogo-A (via the Nogo-66 domain), the proteins and peptides of the invention are useful reagents for detecting Nogo-66 domain-receptor interactions and for screening agents that can inhibit such interactions. Agents (i.e., candidate molecules or drug candidates, which can be proteins, peptides, small organic molecules, nucleic acids, non-organic synthetic molecules, etc.) identified to be able to inhibit interactions or the formation of complexes between Nogo-66 domain-binding receptors (like NgR1) and the proteins/peptides of the invention are potential drug candidates for the therapy of CNS repair and/or neurodegenerative diseases.

In certain embodiments, the invention provides proteins that comprise a mutant human Nogo-66 domain, wherein the cysteine at position 47 (see SEQ ID NO:3) of the human Nogo-66 domain is mutated. Mutating position 47 from a cysteine to another amino acid, for example, valine, prevents Nogo-66 domain-based proteins from aggregating. These mutant Nogo-66 domain-based proteins are able to bind to the NgR1 receptor, and therefore the proteins and peptides of the invention can be used in assays to screen and identify drug candidate compounds that can block, inhibit, prevent or out-compete binding of the Nogo-66 domain to NgR1, thereby indicating that these drug candidates may be potential therapeutic agents for neurodegenerative diseases and neuronal repair.

Nogo-66 Domain

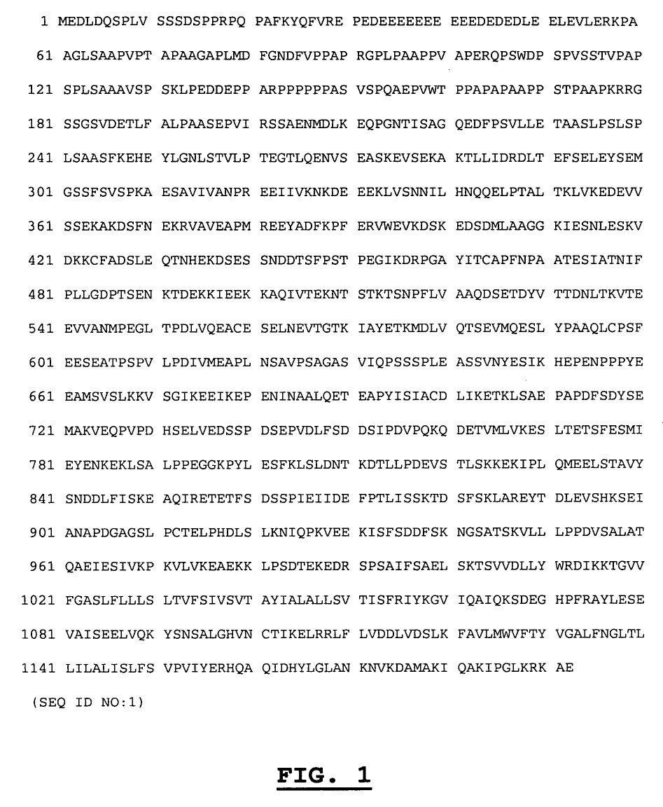

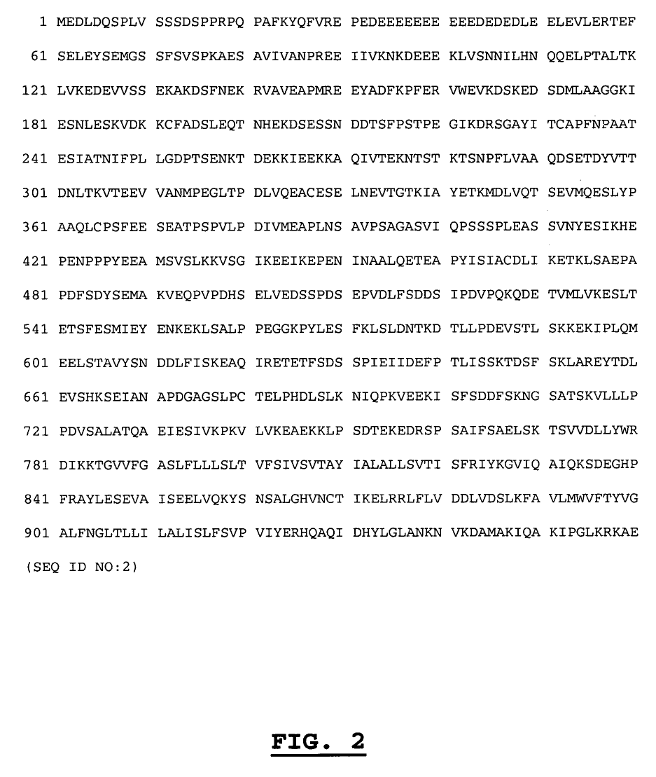

The human Nogo-66 domain comprises amino acid residues 1055-1120 of homo sapiens Nogo-A, where the amino acid sequence for Nogo-A is shown in FIG. 1 and SEQ ID NO:1 (Genbank Accession No. CAB99248). In relation to an alternatively spliced form of homo sapiens Nogo-A (“short form”), the human Nogo-66 domain comprises amino acid residues 823-888, where the Nogo-A short form sequence is shown in FIG. 2 and SEQ ID NO:2 (Genbank Accession No. AAG40878).

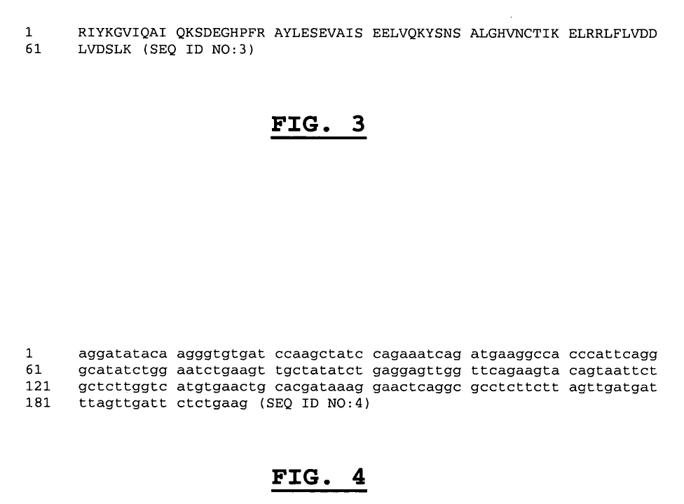

The wild-type amino acid sequence of the human Nogo-66 domain is presented in FIG. 3 and in SEQ ID NO:3. The cysteine residue at position 47 of the Nogo-66 domain can be mutated in order to prevent Nogo-66 domain fusion proteins from aggregating. The nucleotide sequence encoding the human Nogo-66 domain is presented in FIG. 4 and in SEQ ID NO:4. Nucleotides 139-141 of SEQ ID NO:4 encode the cysteine residue of the Nogo-66 domain. In various embodiments, this cysteine residue is mutated (i.e., by mutating one or more of the nucleotides (e.g., nucleotides 139-141 of SEQ ID NO:4) that encode for the cysteine residue) in order to prevent aggregation of the Nogo-66 domain comprising fusion proteins of the invention. These nucleotides can be mutated by standard procedures, for example, by PCR primer-mediated site directed mutagenesis. In one embodiment, the human Nogo-66 domain was mutated such that the cysteine residue was changed to a valine residue, and the amino acid sequence of this particular human Nogo-66 domain mutant is listed in SEQ ID NO:5 (FIG. 5).

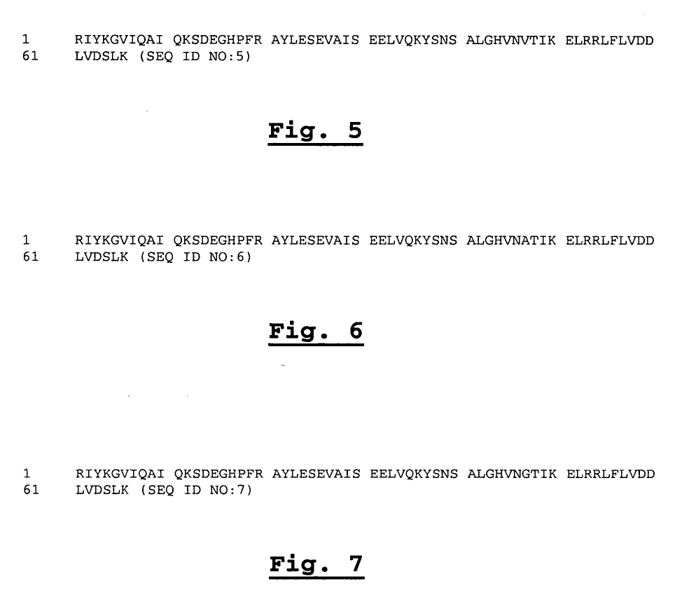

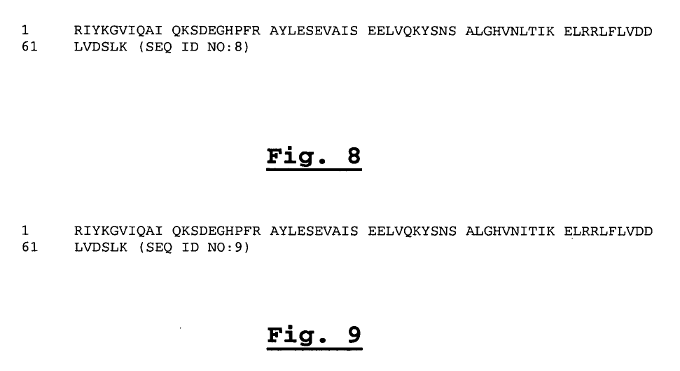

In other embodiments, the human Nogo-66 domain can be mutated such that the cysteine residue is changed to a phenylalanine, leucine, serine, tyrosine, tryptophan, leucine, proline, histidine, glutamine, arginine, isoleucine, methionine, threonine, asparagine, lysine, serine, valine, alanine, aspartic acid, glutamic acid or glycine residue. In one embodiment, the Nogo-66 domain can be mutated such that the cysteine residue is changed to an alanine (SEQ ID NO:6; FIG. 6), glycine (SEQ ID NO:7; FIG. 7), leucine (SEQ ID NO:8; FIG. 8) or isoleucine residue (SEQ ID NO:9; FIG. 9). In another embodiment, the mutation of the human Nogo-66 domain is a deletion of the cysteine residue.

The mutant human Nogo-66 domain comprising proteins and peptides of the invention, whether they are or are not fused to other domains, epitopes, or heterologous sequences, can be used as reagents in a variety of methods. For example, purposes for which the proteins and peptides of the invention can be used include, but are not limited to, as immunogens for the production of antibodies, as reagents to determine whether a receptor can bind to the Nogo-66 domain because the proteins and peptides of the invention have the same or similar ability as the wild-type Nogo-66 domain to bind to receptors, and as reagents to determine whether an agent (i.e., chemical compounds, nucleic acids, proteins, peptides, polymers, etc.) can inhibit or alter the binding between a receptor and a mutant Nogo-66 domain-comprising protein/peptide of the invention.

Reporter Domains

In addition to a human Nogo-66 domain having its cysteine residue mutated to another amino acid, the proteins of the invention also comprise a fusion protein. In one embodiment, the fusion protein comprises a reporter protein domain. The reporter protein domain can be a full-length reporter protein or a domain or portion of a reporter protein. In one embodiment, the reporter protein domain comprises a portion of the human placental secreted alkaline phosphatase protein.

The reporter protein domain can comprise, for example, an enzyme, a fluorescent protein or a luminescent protein, or portions, mutants or variants thereof. The enzyme can be, for example, a human placental alkaline phosphatase protein or a β-galactosidase protein. The fluorescent protein or luminescent proteins include, but are not limited to, luciferase, GFP, red fluorescent protein (RFP), yellow fluorescent protein (YFP), cyan fluorescent protein (CFP), green fluorescent protein (GFP), enhanced version of Green Fluorescent Protein (“eGFP”), blue fluorescent protein (BFP), and sapphire fluorescent protein. The reporter protein domain can be wild-type in genetic sequence or mutated.

Fusion Proteins

The fusion proteins of the invention can comprise at least two elements: (1) a human Nogo-66 domain that is mutated such that it does not possess a cysteine residue, and (2) a reporter protein domain. In addition to these two elements, the fusion proteins can further comprise at least a region coding for a cleavage site for an enzyme or a chemical and/or a region coding for an epitope tag. Further, the fusion proteins can comprise a leader peptide sequence at the 5′ terminus.

In one embodiment, the fusion proteins can comprise an enzyme or chemical cleavage site between the reporter domain and the Nogo-66 domain. When situated between these two domains, the cleavage site enables a user of the invention to separate the two domains if needed. In one embodiment, the region coding for a cleavage site can be an enterokinase protease (EK) cleavage site, where this site comprises the sequence-Asp-Asp-Asp-Lys-(SEQ ID NO: 10). In one embodiment, the EK cleavage site has the sequence-Asp-Asp-Asp-Lys-Leu-Ser-Arg-Asp-(SEQ ID NO:11). Enterokinase is a serine protease that recognizes this amino acid sequence with a high specificity.

Cleavage sites also include, but are not limited to, cleavage sites for the following enzymes and chemicals: Arg-C proteinase, Asp-N endopeptidase, BNPS-Skatole [2-(2-nitrophenyl)-3-methyl-3-bromoindolenine], Caspase, Chymotrypsin, CNBr (cyanogen bromide), Clostripain, Enterokinase, Factor Xa, Formic acid, Glutamyl endopeptidase, Granzyme B, Hydroxylamine, Iodosobenzoic acid, LysC, NTCB (2-nitro-5-thiocyanobenzoic acid), Pepsin, Proline-endopeptidase, Proteinase K, Staphylococcal peptidase I, Thermolysin, Thrombin and Trypsin.

In the following Table, cleavage sites for enzymes and chemicals are presented by position (“P”) with respect to where cleavage occurs in a protein (between P1 and P1). Amino acid positions are numbered with respect to where cleavage occurs, P1 indicates the amino acid immediately 5′ to the cleavage site. P2 indicates the second amino acid immediately 5′ to the cleavage site, and P3, and P4 indicate the third and fourth amino acid immediately 5′ to the cleavage site, respectively. Similarly, P1′ and P2′ indicate the first and second amino acids immediately 3′ to the cleavage site. Where a dash symbol is presented, this indicates that any amino acid can be present.

TABLE 1
Cleavage Sites

| Enzyme name | P4 | P3 | P2 | P1 | P1′ | P2′ |
|---|---|---|---|---|---|---|
| Arg-C proteinase | — | — | — | R | — | — |
| Asp-N endopeptidase | — | — | — | — | D | — |
| BNPS-Skatole | — | — | — | W | — | — |
| Caspase 1 | F, W, Y, or L | — | H, A or T | D | not E, D, Q, | — |
| | | | | | K or R | |
| Caspase 2 | D | V | A | D | not P, E, D, | — |
| | | | | | Q, K or R | |
| Caspase 3 | D | M | Q | D | not P, E, D, | — |
| | | | | | Q, K or R | |
| Caspase 4 | L | E | V | D | not P, E, D, | — |
| | | | | | Q, K or R | |
| Caspase 5 | L or W | E | H | D | — | — |
| Caspase 6 | V | E | H or I | D | not P, E, D, | — |
| | | | | | Q, K or R | |
| Caspase 7 | D | E | V | D | not P, E, D, | — |
| | | | | | Q, K or R | |
| Caspase 8 | I or L | E | T | D | not P, E, D, | — |
| | | | | | Q, K or R | |
| Caspase 9 | L | E | H | D | — | — |
| Caspase 10 | I | E | A | D | — | — |
| Chymotrypsin-high specificity | — | — | — | F or Y | not P | — |
| (C-term. to [FYW], not before P) | — | — | — | W | not M or P | — |
| Chymotrypsin-low specificity | — | — | — | F, L or Y | not P | — |
| (C-term. to [FYWML], not before P) | — | — | — | W | not M or P | — |
| | — | — | — | M | not P or Y | — |
| | — | — | — | H | not D, M, P or W | — |
| Clostripain (Clostridiopeptidase B) | — | — | — | R | — | — |
| CNBr | — | — | — | M | — | — |
| Enterokinase | D or N | D or N | D or N | K | — | — |
| Factor Xa | A, F, G, I, L, | D or E | G | R | — | — |
| | T, V or M | | | | | |
| Formic acid | — | — | — | D | P | — |
| Glutamyl endopeptidase | — | — | — | E | — | — |
| GranzymeB | I | E | P | D | — | — |
| Hydroxylamine | — | — | — | N | G | — |
| Iodosobenzoic acid | — | — | — | W | — | — |
| LysC | — | — | — | K | — | — |
| NTCB (2-nitro-5-thiocyanobenzoic acid) | — | — | — | — | C | — |
| Pepsin (pH 1.3) | — | not H, K, or R | not P | not R | F, L, W or Y | not P |
| | — | not H, K, or R | not P | F, L, W or Y | — | not P |
| Pepsin (pH > 2) | — | not H, K or R | not P | not R | F or L | not P |
| | — | not H, K or R | not P | F or L | — | not P |
| Proline-endopeptidase | — | — | H, K or R | P | not P | — |
| Proteinase K | — | — | — | A, E, F, I, L, | — | — |
| | | | | T, V, W or Y | | |
| Staphylococcal peptidase I | — | — | not E | E | — | — |
| Thermolysin | — | — | — | not D or E | A, F, I, L, M or V | — |
| Thrombin | — | — | G | R | G | — |
| | A, F, G, I, L, | A, F, G, I, L, | P | R | not D or E | not DE |
| | T, V or M | T, V, W or A | | | | |
| Trypsin (exceptions noted below) | — | — | — | K or R | not P | — |
| | — | — | W | K | P | — |
| | — | — | M | R | P | — |

The above cleavage rules do not apply for Trypsin, i.e. no cleavage occurs, with the following compositions of the cleavage sites:

| Enzyme name | P4 | P3 | P2 | P1 | P1′ | P2′ |
|---|---|---|---|---|---|---|
| Trypsin | — | — | C or D | K | D | — |
| | — | — | C | K | H or Y | — |
| | — | — | C | R | K | — |
| | — | — | R | R | H or R | — |

The fusion proteins of the invention can include an epitope tag. An epitope is a portion of a molecule to which an antibody binds. Epitopes can be composed of sugars, lipids or amino acids. In most cases, epitope tags are constructed of amino acids. Epitope tags are added to a molecule (usually proteins) to provide a mechanism for visualization and/or purification. Visualization can take place in a gel, a western blot or labeling via immunofluorescence. Purification can occur by chromatography. An epitope tag can be placed anywhere within the fusion protein, but typically it is placed on either the amino or carboxyl terminus to minimize any potential disruption in tertiary structure and thus function. Although any short stretch of amino acids known to bind an antibody could become an epitope tag, specific epitope tags include, but are not limited to, c-Myc, HA and His₆. The c-Myc epitope is a 10-amino acid segment of the human protooncogene myc (EQKLISEEDL (SEQ ID NO: 12). The HA epitope is an amino acid segment of the haemoglutinin protein from human influenza hemagglutinin protein (YPYDVPDYA (SEQ ID NO:13). The His₆ epitope is composed of six consecutive histidines (HHHHHH (SEQ ID NO:14)). If six histidines are placed in a row, they form a structure that binds the element Nickel, which is especially useful for affinity chromatography-based purification.

The reporter domain in the fusion proteins can also be an epitope tag. There are commercially available antibodies to most of the reporter proteins mentioned above, and these antibodies can be used in a variety of applications, including detection and purification.

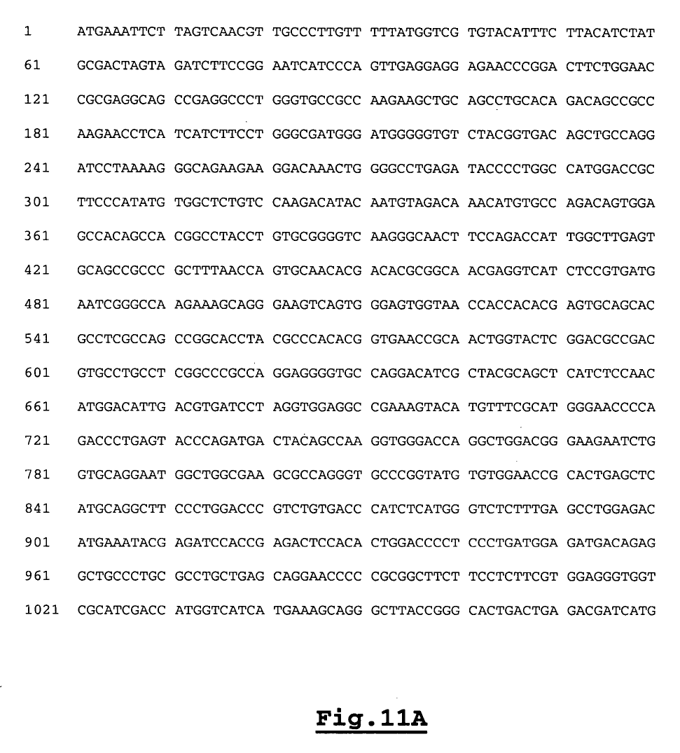

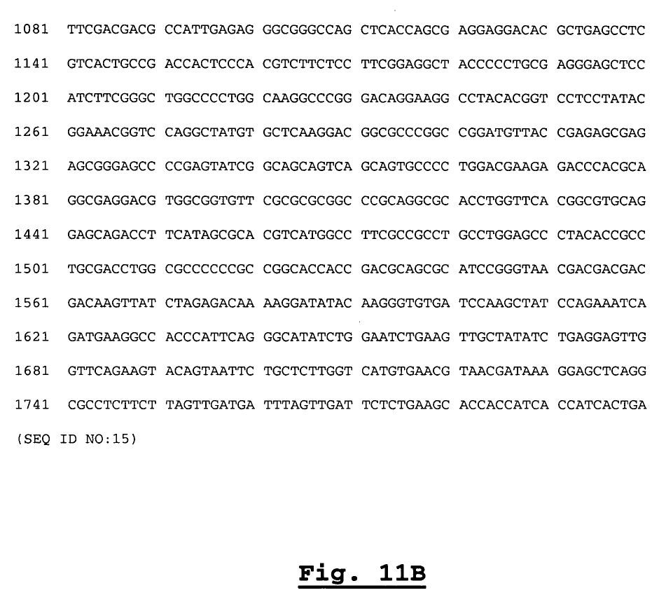

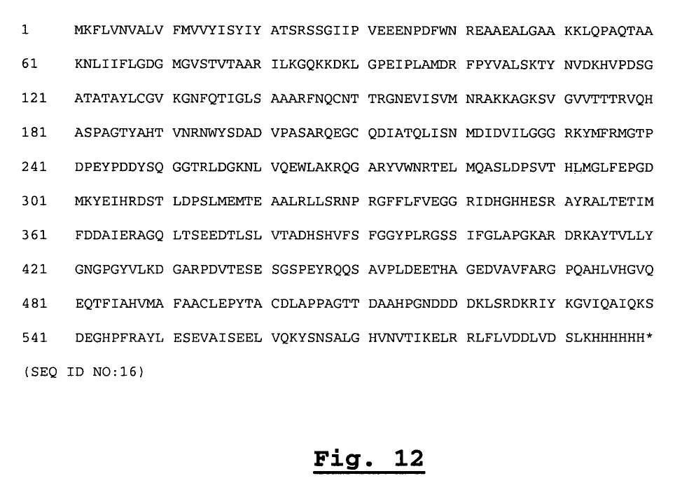

In one embodiment, a fusion protein of the invention is encoded by the nucleotide sequence of SEQ ID NO:15 (FIGS. 11A-B), and therefore has the amino acid sequence of SEQ ID NO:16 (FIG. 12). The amino acid sequence of SEQ ID NO:16 includes the honey bee melatonin signal peptide (amino acids 1-21 of SEQ ID NO:16), a portion of the human placental secreted alkaline phosphatase protein (amino acids 28-516 of SEQ ID NO:16), an enterokinase (EK) protease cleavage site (amino acids 518-526 of SEQ ID NO:16), a human Nogo-66 domain having its cysteine mutated to a valine (amino acids 528-593 of SEQ ID NO:16, where the valine in place of a cysteine is at amino acid position 574), and a His₆ epitope tag (amino acids 594-599 of SEQ ID NO:16). Other leader peptides or signal peptides other than the honey bee melatonin signal peptide can be used in the fusion proteins of the invention known to one skilled in the art.

Purification/Isolation of Proteins and Peptides of the Invention

A cDNA coding for a protein of the invention can be inserted into an expression vector in order to express and purify the protein. For example, the cDNA sequence (SEQ ID NO:15) encoding the fusion protein of SEQ ID NO:16 can be inserted into the pSMED2 mammalian expression vector. Such mammalian expression vectors can be transfected into appropriate cells, such as HEK293 cells. For proteins that are secreted from cells (such as the fusion protein of SEQ ID NO:16), the medium in which the cells are grown is collected in order to purify the protein (see Examples 1 and 2).

Alternatively, when the invention provides a protein that is not secreted from a cell, the protein can be purified from cell lysates. After transfection of a cell with an expression vector containing the coding sequence for the protein, the cells can be isolated such that protein extracts can be made from the cells. The protein extracts can then be subjected to column-chromatography such that the protein can be purified. The protein can be isolated by chromatography by standard procedures known to one skilled in the art. For example, if the protein contains a 6-His tag, the fusion protein can be purified metal affinity chromatography.

Methods for Detecting Receptor/Ligand Interactions and Agents that can Block Or Disrupt Such Interactions

The mutant human Nogo-66 domain comprising proteins and peptides of the invention, whether they are or are not fused to other domains, epitopes, or heterologous sequences, can be used, for example, as reagents to determine whether a receptor can bind to the Nogo-66 domain and as reagents to determine whether an agent can inhibit or alter the binding between a receptor and a mutant Nogo-66 domain-comprising protein/peptide of the invention. The proteins and peptides of the invention can be used as reagents to determine whether a receptor can bind to the Nogo-66 domain because the mutation of the cysteine residue in the Nogo-66 domain does not or does not significantly affect its binding ability to receptors that can bind to the wild-type Nogo-66 domain. Thus, a determination that a protein/peptide of the invention can bind to a receptor indicates that the receptor can most likely bind to the wild-type Nogo-66 domain (and therefore the Nogo-A protein).

Further, the proteins and peptides of the invention can be used to screen for agents that can block, disrupt, inhibit, or out-compete binding between receptors and the Nogo-66 domain (and proteins that contain the Nogo-66 domain). For example, because it is known that the NgR1 receptor binds to the Nogo-A protein, which contains the Nogo-66 domain, the proteins/peptides of the invention can bind to the NgR1 receptor. Agents, such as chemical compounds, nucleic acids, proteins, peptides, and polymers (also referred to herein as “drug candidates” or “candidate molecules”), can be incubated with Nogo-66 domain binding receptors. If the incubation of an agent with the receptor prevents binding of the receptor with a protein/peptide of the invention, then this indicates that the agent itself can specifically bind to the receptor such that the agent can be a potential candidate molecule for use in the treatment of neurodegenerative diseases or CNS damage. The incubation of the agent with the receptor can occur prior to, at the same time, or after contacting the receptor with a protein/peptide of the invention.

Assaying for whether there is an interaction between protein/peptide of the invention and a receptor (and therefore for assaying whether an agent or candidate molecule can inhibit, block, disrupt, out-compete or otherwise alter binding between a receptor and a protein/peptide of the invention) can be conducted by standard protein biochemical techniques known to one skilled in the art. Whether the assay is cell-free (where a receptor can be affixed in a column, resin, beads, etc.) or cell-based (where the receptor is expressed on the surface of a cell, and the protein/peptide of the invention is added to the cell-culture), after contacting a receptor with the protein/peptide of the invention, the sample is washed to remove any protein/peptide that is unbound to the receptor. The sample can then be screened by standard protein assays to determine whether the sample contains the protein/peptide of the invention. Standard protein assays include, but are not limited to, immunoblotting, gel electrophoresis of proteins, ion-exchange chromatography, immunoaffinity chromatography, metal-chelate affinity chromatography, high pressure liquid chromatography, immunoprecipitation, and combinations thereof (see Short Protocols in Molecular Biology, Fifth Ed., Eds. Frederick M. Ausubel et al., John Wiley & Sons, Inc., 2002).

In one embodiment, an interaction between a protein/peptide of the invention and a receptor can be assayed by immunoblotting, where the immunodetection is conducted with an antibody against the Nogo-66 domain. If the protein/peptide of the invention comprises a heterologous epitope, such as an HA-tag or His₆ tag, then the immunoblotting and immunodetection can be conducted with antibodies against the heterologous epitope. As used herein, a heterologous epitope includes epitopes from reporter domains that may be present in a fusion protein of the invention. Therefore, antibodies against reporter domains can also be used in immunoblotting assays. In other embodiments, where the protein/peptide of the invention comprises a reporter domain, an interaction between the protein/peptide of the invention and a receptor can be assayed for by detecting whether fluorescence or luminescence is emitted from the sample.

In one embodiment, the invention provides a method for detecting whether a receptor can bind to a Nogo-66 domain, the method comprising: (a) contacting a receptor with a protein or peptide comprising a mutant human Nogo-66 domain, wherein the mutant human Nogo-66 domain does not comprise a cysteine residue; and (b) assaying for the presence of a protein complex comprising the receptor and the protein or peptide comprising a mutant human Nogo-66 domain. If a protein complex of the receptor and the protein/peptide comprising a mutant human Nogo-66 domain is detected, this indicates that the receptor can bind to the Nogo-66 domain.

In another embodiment, the invention provides a method for detecting whether a receptor can bind to a Nogo-66 domain, the method comprising: (a) incubating (i) a receptor and (ii) a fusion protein of the invention that comprises a reporter domain; and (b) assaying for reporter protein activity from the well, wherein detection of reporter protein activity indicates that the receptor can bind to the Nogo-66 domain.

For all the methods of the invention, the receptor that is incubated can be a receptor expressed on the surface of a cell, and thus, the cell itself can be incubated. Further, the receptor that is expressed by the cell can be encoded by an expression vector that has been transfected into the cell. In another embodiment, the expression vector can be from an expression vector library, which when used in the present methods, allows for high-throughput screening for receptors that are capable of binding to the Nogo-66 domain.

In another embodiment, the invention provides a method for detecting whether a candidate molecule can inhibit binding between Nogo-66 and a receptor, the method comprising: (a) contacting a receptor that binds to a Nogo-66 domain with: (i) a protein or peptide comprising a mutant human Nogo-66 domain, wherein the mutant human Nogo-66 domain does not comprise a cysteine residue, and (ii) a candidate molecule; and (b) assaying for the presence of a protein complex comprising the receptor and the protein or peptide comprising a mutant human Nogo-66 domain. If a protein complex of the receptor and the protein/peptide comprising a mutant human Nogo-66 domain (i.e., the Nogo-66 domain does not comprise a cysteine residue such that protein/peptide does not aggregate) is not detected, or if lower amounts of the complex is detected as compared to samples where the candidate molecule was not added, this indicates that the candidate molecule can inhibit, block, disrupt, out-compete or otherwise alter protein interaction or binding between the receptor and the Nogo-66 domain. The receptor can be, for example, the NgR1 protein.

To screen drug candidates in a high-throughput manner, the ability of drug candidates to block NgR1 protein-protein interactions can be tested by detecting fluorescence or luminescence. As used herein, the term “drug candidates” is not meant to be limiting, and can include any chemical or biological compound, including small-molecules, nucleic acids, proteins, peptides, and synthetic entities, including nanospheres. Fluorescent or luminescent fusion proteins can be made with the Nogo66 domain, as described above, and/or with the NgR1 protein.

In one embodiment, a method for screening drug candidates that can block NgR1 interaction to a Nogo-66 domain comprises assaying whether a drug candidate can block an alkaline phosphatase Nogo-66 domain reporter fusion protein interaction with NgR1. For example, in Example 3, cells expressing NGR1 are incubated with an alkaline phosphatase comprising fusion protein (such as SEQ ID NO:16) in multi-well tissue culture plates. If no agent is added to the wells that can block the interaction between the Nogo-66 domain of the fusion protein and NgR1, then when a chemiluminescent or fluorescent substrate is added to the wells, the alkaline phosphatase domain of the fusion protein will react with the substrate such that the wells provide chemiluminescence of fluorescence that can be detected by a luminometer or fluorometer. However, if a drug candidate is added to a well that can inhibit or block the interaction between the Nogo-66 domain of the fusion protein and NgR1, then the majority of the fusion protein added to each well is prevented from binding to NgR1. Thus, the majority of the fusion protein will be washed away in the assay (see Example 3) prior to the addition of chemiluminescent or fluorescent substrates to the wells. The wells containing drug candidates that can block Nogo-66 domain and NgR1 interactions will not provide chemiluminescent or fluorescent signals (or lower signals as compared to wells not containing the drug candidates, i.e., negative control wells with no drug candidates added). These drug candidates can then be further tested for their ability to help regenerate damaged axons after traumatic injury, stroke, or certain neurodegenerative diseases.

Suitable substrates for alkaline phosphatase include, but are not limited to, p-nitrophenyl phosphate, D-luciferin-O-phosphate or a substituted 1,2-dioxetane-phosphate. Substituted 1,2-dioxetane-phosphates include, for example, CSPD (3-(4-methoxyspiro-{1,2-dioxetane-3,2′-(5′-chloro)tricyclo[3.3.1.1^{3,7}]decan}-4-yl)phenyl phosphate), AMPPD or Lumigen-PPD (disodium 3-(4-methoxyspiro{1,2-dioxetane-3,2′-tricyclo[3.3.1.1^{3,7}]decan}-4-yl) phenyl phosphate), and AMPPD with substituted chlorine moiety on adamantine ring.

In one embodiment, a method for screening drug candidates that can block NgR1 interaction to a Nogo-66 domain comprising protein comprises a cell-based method. For example, a Nogo-66-fluorescent fusion protein and an NgR1-fluorescent fusion protein can be expressed in cells that do not endogenously express the proteins, such as 293 cells. In the absence of any agent that can block the Nogo-66 domain from binding to NgR1, then the cells should have significant co-localization of two fluorescent color emissions. For example, Nogo-66-domain-GFP binding to NgR1-YFP should present co-localized fluorescence of yellow and green fluorescence. Drug candidates can then be added to cells that express the fluorescent fusion proteins to test for inhibition of co-localized fluorescence. This can be conducted in a high-throughput manner, where microplates (for example, 96-well, 384-well, or 1536-well plates) are used for incubating the cells and each individual well can contain a different drug candidate.

Co-localized fluorescence or the decrease of co-localized fluorescence can be detected by confocal fluorescence coincidence analysis. Confocal fluorescence coincidence analysis extracts fluorescence fluctuations that occur coincidently in two different spectral ranges. This procedure makes it possible to monitor whether an association between molecular fragments that are labeled with different fluorophores is established or broken. Confocal fluorescence coincidence analysis is a very sensitive and ultrafast technique with readout times of 100 ms and below.

Alternatively, an inhibition of Nogo-66 domain protein interaction with NgR1 can be detected by fluorescence resonance emission transfer (FRET) detection or time-resolved FRET (TR-FRET) detection. FRET has been established as a sensor of protein-protein interactions (Miyawaki et al. (1997), Nature, 388: 882-887). When two proteins comprising fluorescent protein domains interact, exciting light causes one of the fluorescent protein domains (fluorophore donor) to transfer its energy to a second, longer wavelength fluorophore (fluorophore acceptor) in a non-radiative manner. When there is a transfer of excitation energy from the donor to the acceptor, there is no emission by the donor. Therefore, when a drug candidate inhibits Nogo-66/NgR1 protein-protein interactions, the drug candidate disrupts FRET energy transmission such that the donor fluorophore does emit its fluorescence. In other words, when a drug candidate inhibits Nogo-66/NgR1 protein-protein interactions, two types of fluorescence are detected. If the drug candidate cannot inhibit Nogo-66/NgR1 protein interactions, then only one type of fluorescence is detected, as fluorescence energy emission transfer is not inhibited. Fluorescence energy emission transfer is distance dependent; two fluorophores have to be in sufficient proximity (less than 100 angstroms) such that an excited fluorophore can transfer its energy to a second fluorophore.

In another embodiment, a method for screening whether a drug candidate can inhibit Nogo-66/NgR1 protein-protein interactions can comprise bioluminescence resonance energy transmission (BRET). In BRET, there is a luminescent donor protein and a fluorescent acceptor protein. For example, the luminescent donor protein can comprise a Nogo-66 domain fused to luciferase and the fluorescent acceptor protein can comprise an NgR1 protein or domain thereof fused to yellow fluorescent protein. In most applications the fused donor is Renilla luciferase (Rluc) and the acceptor is a fusion with YFP (to increase the spectral distinction between the two emissions). When the donor and acceptor are in close proximity, i.e., when the two fusion proteins interact, the energy resulting from catalytic degradation of a coelenterazine derivative substrate is transferred from the luciferase to the YFP, which will then emit fluorescence at its characteristic wavelength. Thus, when the two fusion proteins interact, there are two signals, luciferase and YFP. When the two fusion proteins do not interact, for example, when a drug candidate inhibits their interaction, then there is only one signal that can be detected, the excited luciferase.

As various changes can be made in the above methods and compositions without departing from the scope and spirit of the invention as described, it is intended that all subject matter contained in the above description, shown in the accompanying drawings, or defined in the appended claims be interpreted as illustrative, and not in a limiting sense.

### EXAMPLES OF THE INVENTION

The following examples are representative of techniques employed by the inventors in carrying out aspects of the present invention. It should be appreciated that while these techniques are exemplary for the practice of the invention, those of skill in the art, in light of the present disclosure, will recognize that numerous modifications can be made without departing from the spirit and intended scope of the invention. Thus, the examples described below are provided to illustrate the present invention and are not included for the purpose of limiting the invention.

### Example 1

### Mutant Nogo-66 Fusion Protein

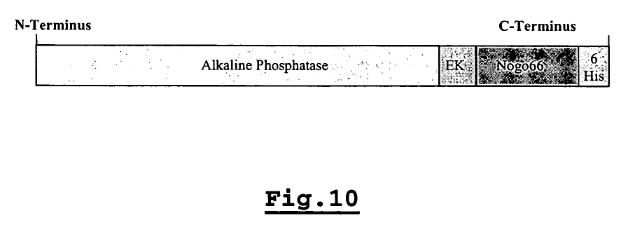

To facilitate the study of Nogo-66 interaction with its neuronal receptors, and to explore potential therapeutic opportunities, a recombinant human Nogo-66 fusion protein was constructed (for a basic schematic of the fusion protein, see FIG. 10). The fusion contained a honey bee melatonin signal peptide at its N-terminus, a portion of the human placental secreted alkaline phosphatase protein, an enterokinase (EK) cleavage site, a mutant Nogo-66 coding region (SEQ ID NO:5), and a region coding for six consecutive histidines (6 His-tag or His₆). The nucleotide sequence coding for the fusion protein is listed in SEQ ID NO:15, and the amino acid sequence of the fusion protein is listed in SEQ ID NO:16.

The mutant Nogo-66 coding region contains a mutation where the cysteine residue at amino acid position 47 of the wild-type Nogo-66 domain peptide is replaced by valine. This mutation was generated by oligonucleotide site directed mutagenesis. Briefly, a sample reaction mixture was prepared as follows: 5 μl of 10× reaction buffer from the QuickChange® XL site-directed mutagenesis kit from Stratagene (La Jolla, Calif.); 2 μl (10 ng) of dsDNA template (i.e., DNA comprising the coding sequence for wild-type human Nogo-66 domain in a vector); 1.25 μl (125 ng) of a sense-strand oligonucleotide primer containing the coding sequence for the amino acid desired to replace the cysteine residue (for example, 5′-CTGCTCTTGGTCATGTGAACGTAACGATAAAGGAGCTCAGGCG-3′ (SEQ ID NO:17), where the bolded-underlined nucleotides “GTA” encode the valine that is to replace the cysteine residue); 1.25 μl (125 ng) of a antisense-strand oligonucleotide primer (for example, 5′-CGCCTGAGCTCCTTTATCGTTACGTTCACATGACCAAGAGCAG-3′ (SEQ ID NO:18); 1 μl of dNTP mix, 3 μl of QuikSolution, ddH₂O 36.5 μl to a final volume of 50 μl. To the sample reaction mix, 1 μl of Pfu Turbo DNA polymerase (2.5 U/μl) was added to each sample reaction. PCR was then conducted on the mixture for 1 cycle of 95° C. for 1 minute, 18 cycles of 95° C. for 50 seconds, 60° C. for 50 seconds, and 68° C. for 7.5 minutes, and 1 cycle of 68° C. for 7 minutes. After PCR, the PCR samples were digested with Dpn I, which digests nonmutated Nogo-66 domain DNA. The digested PCR samples were then transformed into bacterial cells for DNA preparation.

The cDNA (SEQ ID NO:15) of the full-length fusion protein was inserted into the pSMED2 mammalian expression vector and the predicted sequence was confirmed by standard means. The vector was transfected into HEK293 cells and after 48 hours the medium in which the cells were grown was collected (referred to as conditioned medium) (see Example 2). After transfection, the conditioned medium was assayed to determine whether the fusion protein was secreted from the cells. The presence of the fusion protein in the conditioned medium was verified by Western blotting using an antibody against the alkaline phosphatase portion of the fusion. The fusion protein was purified from conditioned medium by TALON (BD/Clonetech) metal affinity chromatography (via the 6 His-tag of the fusion protein) (see Example 2). The purified protein was found to migrate at the predicted molecular weight by polyacrylamide gel electrophoresis. No bands of higher molecular weight were identified, indicating that the protein was not aggregated as had been noted for a similar fusion bearing the wild-type Nogo-66 sequence.

Purified fusion protein was assayed for binding to COS7 cells expressing the human Nogo Receptor-1 protein (NgR1). Binding was confirmed by colorimetric staining of NgR1 expressing cells based on associated alkaline phosphatase activity conferred by the fusion protein (see Example 3). Thus, a non-aggregated fusion protein was created that can be used as a reagent to explore the interaction of Nogo-66 with its receptor, or receptors, as well as to identify agents interfering in that interaction (see Example 3).

### Example 2

### Purification of a Secreted Alkaline Phosphatase Nogo-66 Domain Fusion Reporter Protein

293 HEK cells were transiently transfected with expression vector DNA comprising a secreted alkaline phosphatase mutant Nogo-66 domain fusion reporter protein (i.e., an expression vector containing the coding sequence (SEQ ID NO:15) for the fusion protein of SEQ ID NO:16.

On Day 1: For each transfection, 40 million 293 HEK cells were plated into a p100 plate. Fresh growth medium (DMEM+10% HIFBS+Pen/Strep/Gln) was added leaving 8 ml final volume.

On Day 2: For each p100 plate, 24 μl of Lipofectamine 2000 (Invitrogen, Carlsbad, Calif.) was diluted into 1 ml OptiPRO SFM (Invitrogen). Eight μg of expression vector plasmid was also diluted into 1 ml OptiPRO SFM in a sterile eppendorf. 1 ml of diluted DNA was added to 1 ml of diluted lipofectamine, mixed and incubated at room temperature for 15 minutes. The DNA/lipofectamine mixture was then added to each p100 plate and incubated at 37° C., 5% CO₂ for 16 hours.

On Day 3: The growth medium from cells was removed, and each p110 plate was rinsed with 10 ml sterile PBS. The PBS was removed, and 10 ml of FreeStyle 293 expression medium (Invitrogen) was added to each p100 plate.

On Day 5: The conditioned medium from the plates were harvested (48 hour accumulation). Dead cells were spun-out from the conditioned medium by centrifugation of the medium at 1200 RPM. One tablet of Complete, EDTA-free Protease inhibitor cocktail tablets (Roche, Indianapolis, Ind.) was added into each 50 ml of dead cells-free conditioned medium. The mixture was then put on ice, until metal affinity resin is added to the mixture for fusion protein purification (i.e., purification via the His₆ tag).

The fusion protein was purified from the conditioned medium mixture by using BD TALON Metal Affinity Resins (Becton Dickinson Biosciences, Rockville, Md.). 0.25 ml of TALON resin (for each 10 ml aliquot of conditioned medium) was spun down. The resin was then equilibrated using 1 ml of 1× Equilibration Buffer (5× Equilibration buffer: pH8, 250 mM Sodium Phosphate, 1.5M Sodium Chloride). The resin was spun down again, and was put into the conditioned medium. The tube containing the conditioned medium and the resin was then shaken slowly at 4° C. for 1 hour. After shaking, the resin was spun down and washed three times with 1× wash buffer (5× Wash buffer: pH8, 250 mM Sodium Phosphate, 1.5M Sodium Chloride, 1 mM Imidazole). The fusion protein was then eluted from the resin using 1× Elution buffer (10× Elution Buffer: 1.5 M Imidazole pH7).

### Example 3

### Alkaline Phosphatase Nogo-66 Domain Fusion Reporter Protein Based Binding Assay

The alkaline phosphatase mutant Nogo-66 domain fusion reporter protein (SEQ ID NO:16) does not aggregate. This allows for both ease of purification and for its use in assays to test whether drug candidates can bind to the NgR1 receptor.

A cell-based chemiluminescent SEAP (secreted alkaline phosphastase) assay was used to confirm binding between NgR1 and the fusion reporter protein having the amino acid sequence of SEQ ID NO:16. This high-throughput assay can also be used to determine whether a drug candidate can out-compete or disrupt NgR1 and fusion reporter binding, where if the drug candidate can out-compete or disrupt the binding, then this indicates that the drug candidate can bind to NgR1. The following assay was used to determine that the fusion protein of SEQ ID NO:16 can bind to NgR1. The following assays can be used to determine whether any fusion protein of the invention that contains an alkaline phosphatase domain can bind to NGR1.

Chemiluminescent Assay

On Day 1: CHO cells expressing NGR1 are seeded into each well of a Biocoat poly-D-lysine cellware 96-well plate (Becton Dickinson).

On Day 2:

1. The medium is removed and cells are washed with 200 μl/well of HBAH (Hanks' balanced salt solution, bovine serum albumin at 0.5 mg/ml) at room temperature.

2. 100 μl of conditioned medium containing the fusion protein (i.e., conditioned medium prior to resin purification in Example 2 above) is added (or 2 μg of purified fusion protein can be added) to each well. The wells are incubated at room temperature for 90 minutes.

3. The medium is removed from the wells. The cells are washed six times with 200 μl HBAH at room temperature. For each wash, the HBAH is incubated with the cells for 5 minutes and the plates gently swirled.

4. The HBAH is removed. The cells are then fixed with 200 μl of fixative buffer (acetone-formaldehyde (60%, 3%, 20 mM HEPES, pH 7.0)) for 15 seconds at room temperature.

5. The cells are then washed three times with HBS (Hanks' balanced salt solution), 5 minutes each time at room temperature.

6. Buffers are equilibrated to room temperature. The 5× dilution buffer from the BD Great EscAPe™ SEAP Kit (Becton Dickinson) (“SEAP kit”) is diluted to 1× with ddH₂O.

7. The HBS in the wells is aspirated away. 60 μl of 1× dilution buffer is added to each well.

8. If the conditioned medium containing the fusion protein is to be checked, an aliquot of the medium is diluted one to fifty with 1× dilution buffer. Then 60 μl of this dilution is added to an empty well.

9. If there is a need to check a heated alkaline phosphatase standard curve, 1 μl of placental alkaline phosphatase provided by the SEAP Kit is diluted in 300 μl 1× dilution buffer followed by 1:2 serial dilution for 7 times to give the following standards: 0, 0.3, 0.0625, 0.125, 2.5, 5, 10, 20 ng/60 μl.

10. The sample plates are covered and incubated at 65° C. in a dry air incubator for 90 minutes.

11. The samples are cooled on ice for 2-3 minutes, and then allowed to come to room temperature.

12. If a fresh alkaline phosphatase standard curve is desired, add 60 μl of alkaline phosphatase serial dilution to each empty well (the same as step 9).

13. Add 60 μl of assay buffer (from the SEAP Kit) to each well, and incubate for 5 minutes at room temperature.

14. Dilute CSPD chemiluminescent substrate (from SEAP Kit) 1:20 with chemiluminescent enhancer (from SEAP Kit).

15. Add 60 μl of the diluted substrate to each well, incubate at room temperature for 10 minutes.

16. Read the plates on a LMAXII Luminometer (Molecular Devices, Sunnyvale, Calif.). The signal will be stable for about 10 to 60 minutes. Positive signals indicate the presence of the fusion protein bound to the cells via the NgR1 receptor.

To determine whether a drug candidate can disrupt fusion protein and NgR1 binding, the drug candidate is either pre-incubated with the cells prior to addition of the fusion protein. Drug candidates can be added at amounts to provide final concentrations of the compounds in the wells at 200 μM, 20 μM, or 2 μM (and also a negative control with no drug candidate added) and incubated for about 30 minutes prior to addition of fusion protein. If the drug candidate can block or disrupt or out compete binding of the fusion protein to NgR1, then the well containing the drug candidate will not provide chemiluminescence that can be detected by the luminometer, or the well will provide a lower signal of chemiluminescence as compared to wells that do not contain the same drug candidate.

Fluorescent Assay

Alternatively, a fluorescent based assay can be used to determine whether an alkaline phosphatase containing fusion protein can bind to NgR1 and to determine whether a drug candidate can disrupt such binding. For example, the AttoPhos® AP Fluorescent Substrate System (Promega, Madison, Wis.) provides a highly sensitive fluorescent alkaline phosphatase substrate. The system includes AttoPhos® Substrate, AttoPhos® Buffer and Calibration Solution. AttoPhos® Substrate (2′-[2-benzothiazoyl]-6′-hydroxybenzothiazole phosphate [BBTP]) is cleaved by alkaline phosphatase to produce inorganic phosphate (Pi) and the alcohol, 2′-[2-benzothiazoyl]-6′-hydroxybenzothiazole (BBT).

This enzyme-catalyzed conversion of the phosphate form of AttoPhos® Substrate to BBT is accompanied by an enhancement in fluorescence properties. Relative to AttoPhos® Substrate, BBT has greatly increased quantum efficiency, and fluorescence excitation and emission spectra that are shifted well into the visible region. Relative to other fluorometric reporters, the BBT anion has an unusually large Stokes' shift of 120 nm, which leads to lower levels of background fluorescence and higher detection sensitivity.

Thus, on Day 1: Seed CHO cells expressing NGR1 into wells of a microtest tissue culture plate, 96 well, flat bottom (Becton Dickinson). Cells are about 90-95% confluent the next day.

On Day 2:

1. Remove medium from the wells by gentle aspiration. Add 50 μl HBAH with or without drug candidates to be tested. Drug candidates can be added at amounts to provide final concentrations of the compounds in the wells at 200 μM, 20 μM, or 2 μM (and also a negative control with no drug candidate added) and incubated for about 30 minutes at room temperature prior to addition of fusion protein.

2. Add 50 μl of conditioned medium containing fusion protein (i.e., conditioned medium prior to resin purification in Example 2 above or 2 μg/ml of purified fusion protein in HBAH to the wells. Incubate for another 60 minutes at room temperature on a shaker.

3. Remove buffer by inverting plates on a paper towel, wash the wells 4 times with HBAH.

4. Add 100 μl of AttoPhos at 0.6 mg/ml to each well. Cover plates in aluminum foil. Let the color develop for 30 minutes before reading with a fluorometer.

5. Read the plates at endpoint read at Excitation 435 nm and Emission 555 nm with a FlexStation 384 Microplate Fluorometer (Molecular Devices).

Calculations: (a) The average of the measurements for the wells with CHO cells not expressing NGR1 are defined as background, and this average is subtracted from the raw data for each well; (b) the percent total binding=(background subtracted values) divided by (average of the background subtracted values for wells with no drug candidate added).

What is claimed:

1. A protein or peptide comprising a mutant human Nogo-66 domain, wherein the mutant human Nogo-66 domain does not comprise a cysteine residue.

2. The protein or peptide of claim 1, wherein the protein or peptide does not aggregate.

3. A fusion protein comprising a mutant human Nogo-66 domain, wherein the Nogo-66 domain does not comprise a cysteine residue.

4. A fusion protein comprising:
(a) a mutant human Nogo-66 domain that does not comprise a cysteine residue; and
(b) a reporter protein domain.

5. The protein of claim 1, 3 or 4, wherein the mutant human Nogo-66 domain comprises an amino acid sequence of SEQ ID NO:5, 6, 7, 8 or 9, or an amino acid sequence that is at least 95% identical to SEQ ID NO:5, 6, 7, 8 or 9.

6. The protein of claim 1, 3 or 4, wherein the mutant human Nogo-66 domain has an amino acid sequence of SEQ ID NO:5 or an amino acid sequence that is at least 95% identical to SEQ ID NO:5.

7. The protein of claim 4, wherein the reporter protein domain comprises an enzyme, a luminescent protein, or a fluorescent protein.

8. A fusion protein comprising:
(a) a mutant human Nogo-66 domain having the amino acid sequence of SEQ ID NO:5; and
(b) an alkaline phosphatase domain.

9. A fusion protein comprising the amino acid sequence of SEQ ID NO:16.

10. A method for detecting whether a receptor can bind to a Nogo-66 domain, the method comprising:
(a) contacting a receptor with a protein according to claim 1 or 4; and
(b) assaying for a protein complex comprising the receptor and the protein according to claim 1 or 4.

11. A method for testing whether a molecule can inhibit binding between a Nogo-66 domain and a receptor, the method comprising:
(a) contacting a receptor with:
(i) a protein according to claim 1 or 4, and
(ii) a candidate molecule; and
(b) assaying for a protein complex comprising the receptor and the protein according to claim 1 or 4, thereby testing whether the candidate molecule can inhibit binding between the Nogo-66 domain and the receptor.

12. The method of claim 10, wherein in step (a) the receptor is contacted with a fusion protein; and wherein in step (b) the assaying for the protein complex comprises detecting reporter protein activity from the fusion protein.

13. The method of claim 11, wherein in step (a) the receptor is contacted with a fusion protein; and wherein in step (b) the assaying for the protein complex comprises detecting reporter protein activity from the fusion protein.

14. The method of claim 10, wherein the receptor is expressed on the surface of a cell.

15. The method of claim 11, wherein the receptor is expressed on the surface of a cell.

16. The method of claim 10, wherein the receptor is NgR1.

17. The method of claim 11, wherein the receptor is NgR1.

18. The method of claim 11, wherein the method is conducted in a high-throughput manner.

19. The method of claim 18, wherein the method is conducted in a 96-well, a 384-well or a 1536-well microplate.

20. The method of claim 11, wherein the candidate molecule is from a small-molecule chemical library.

21. The method of claim 11, wherein the candidate molecule comprises a peptidomimetic molecule that mimics the binding of Nogo-66 to a Nogo receptor.

22. The method of claim 11, wherein the candidate molecule is from a peptide library.

23. The method of claim 10, wherein the receptor is expressed on the surface of a cell, and wherein the receptor is encoded by a cDNA library expression vector that has been transfected into the cell.

24. The method of claim 12, wherein the fusion protein comprises a reporter protein domain that comprises a human placental alkaline phosphatase protein.

25. The method of claim 12, wherein the fusion protein comprises the amino acid sequence of SEQ ID NO:16.

26. A method for testing whether a molecule can inhibit binding between a Nogo-66 domain and a receptor, the method comprising:
(a) incubating in a first well:
(i) a cell expressing a receptor that binds to Nogo-66,
(ii) a fusion protein according to claim 4, and
(iii) a candidate molecule;
(b) incubating in a second well:
(i) a cell expressing the receptor that binds to Nogo-66, and
(ii) the fusion protein used in step (a); and
(c) assaying for reporter protein activity from the first and second well, wherein detection of decreased of reporter protein activity in the first well as compared to the second well indicates that the candidate molecule can inhibit the binding between the receptor and the Nogo-66 domain.

27. The method of claim 26, wherein the method is conducted in a high-throughput manner.

28. The method of claim 27, wherein the method is conducted in a 96-well, a 384-well or a 1536-well microplate.

29. The method of claim 26, wherein the candidate molecule is from a small-molecule chemical library.

30. The method of claim 26, wherein the candidate molecule comprises a peptidomimetic molecule that mimics the binding of Nogo-66 to a Nogo receptor.

31. The method of claim 26, wherein the candidate molecule is from a peptide library.

32. The method of claim 26, wherein the fusion protein comprises a reporter protein domain that comprises a human placental alkaline phosphatase protein.

33. The method of claim 26, wherein the fusion protein comprises SEQ ID NO:16.

34. A method for testing whether a molecule can inhibit binding between a Nogo-66 domain and NGR1, the method comprising:
(a) incubating in a first well:
(i) a cell expressing the NgR1 protein on its cell surface,
(ii) a fusion protein of claim 9, and
(iii) a candidate molecule;
(b) incubating in a second well:
(i) a cell expressing the NgR1 protein on its cell surface, and
(ii) a fusion protein of claim 9; and
(c) assaying for reporter protein activity from the first and second well, wherein detection of decreased of reporter protein activity in the first well as compared to the second well indicates that the candidate molecule can inhibit the binding between the receptor and the Nogo-66 domain.

35. The method of claim 34, wherein assaying comprises adding an alkaline phosphatase substrate to the first and second wells.


SEQUENCE LISTING (18 sequences)

SEQ ID NO: 1 - protein, 1192 aa, Homo sapiens [sequence omitted]

SEQ ID NO: 2 - protein, 960 aa, Homo sapiens
MEDLDQSPLVSSSDSPPRPQPAFKYQFVREPEDEEEEEEEEEEDEDEDLEELEVLERTEFSELEYSEMGSSFSVSPKAESAVIVANPREEIIVKNKDEEEKLVSNNILHNQQELPTALTKLVKEDEVVSSEKAKDSFNEKRVAVEAPMREEYADFKPFERVWEVKDSKEDSDMLAAGGKIESNLESKVDKKCFADSLEQTNHEKDSESSNDDTSFPSTPEGIKDRSGAYITCAPFNPAATESIATNIFPLLGDPTSENKTDEKKIEEKKAQIVTEKNTSTKTSNPFLVAAQDSETDYVTTDNLTKVTEEVVANMPEGLTPDLVQEACESELNEVTGTKIAYETKMDLVQTSEVMQESLYPAAQLCPSFEESEATPSPVLPDIVMEAPLNSAVPSAGASVIQPSSSPLEASSVNYESIKHEPENPPPYEEAMSVSLKKVSGIKEEIKEPENINAALQETEAPYISIACDLIKETKLSAEPAPDFSDYSEMAKVEQPVPDHSELVEDSSPDSEPVDLFSDDSIPDVPQKQDETVMLVKESLTETSFESMIEYENKEKLSALPPEGGKPYLESFKLSLDNTKDTLLPDEVSTLSKKEKIPLQMEELSTAVYSNDDLFISKEAQIRETETFSDSSPIEIIDEFPTLISSKTDSFSKLAREYTDLEVSHKSEIANAPDGAGSLPCTELPHDLSLKNIQPKVEEKISFSDDFSKNGSATSKVLLLPPDVSALATQAEIESIVKPKVLVKEAEKKLPSDTEKEDRSPSAIFSAELSKTSVVDLLYWRDIKKTGVVFGASLFLLLSLTVFSIVSVTAYIALALLSVTISFRIYKGVIQAIQKSDEGHPFRAYLESEVAISEELVQKYSNSALGHVNCTIKELRRLFLVDDLVDSLKFAVLMWVFTYVGALFNGLTLLILALISLFSVPVIYERHQAQIDHYLGLANKNVKDAMAKIQAKIPGLKRKAE

SEQ ID NO: 3 - protein, 66 aa, Homo sapiens
RIYKGVIQAIQKSDEGHPFRAYLESEVAISEELVQKYSNSALGHVNCTIKELRRLFLVDDLVDSLK

SEQ ID NO: 4 - DNA, 198 bp, Homo sapiens
aggatatacaagggtgtgatccaagctatccagaaatcagatgaaggccacccattcagggcatatctggaatctgaagttgctatatctgaggagttggttcagaagtacagtaattctgctcttggtcatgtgaactgcacgataaaggaactcaggcgcctcttcttagttgatgatttagttgattctctgaag

SEQ ID NO: 5 - protein, 66 aa, Homo sapiens
RIYKGVIQAIQKSDEGHPFRAYLESEVAISEELVQKYSNSALGHVNVTIKELRRLFLVDDLVDSLK

SEQ ID NO: 6 - protein, 66 aa, Homo sapiens
RIYKGVIQAIQKSDEGHPFRAYLESEVAISEELVQKYSNSALGHVNATIKELRRLFLVDDLVDSLK

SEQ ID NO: 7 - protein, 66 aa, Homo sapiens
RIYKGVIQAIQKSDEGHPFRAYLESEVAISEELVQKYSNSALGHVNGTIKELRRLFLVDDLVDSLK

SEQ ID NO: 8 - protein, 66 aa, Homo sapiens
RIYKGVIQAIQKSDEGHPFRAYLESEVAISEELVQKYSNSALGHVNLTIKELRRLFLVDDLVDSLK

SEQ ID NO: 9 - protein, 66 aa, Homo sapiens
RIYKGVIQAIQKSDEGHPFRAYLESEVAISEELVQKYSNSALGHVNITIKELRRLFLVDDLVDSLK

SEQ ID NO: 10 - protein, 4 aa, Artificial
  Enterokinase cleavage motif
DDDK

SEQ ID NO: 11 - protein, 8 aa, Artificial
  Enterokinase cleavage motif
DDDKLSRD

SEQ ID NO: 12 - protein, 10 aa, Homo sapiens
EQKLISEEDL

SEQ ID NO: 13 - protein, 9 aa, Homo sapiens
YPYDVPDYA

SEQ ID NO: 14 - protein, 6 aa, Artificial
  Protein purification sequence
HHHHHH

SEQ ID NO: 15 - DNA, 1800 bp, Artificial
  Fusion of Homo sapiens and non-Homo sapiens sequences
atgaaattcttagtcaacgttgcccttgtttttatggtcgtgtacatttcttacatctatgcgactagtagatcttccggaatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgcagcgcatccgggtaacgacgacgacgacaagttatctagagacaaaaggatatacaagggtgtgatccaagctatccagaaatcagatgaaggccacccattcagggcatatctggaatctgaagttgctatatctgaggagttggttcagaagtacagtaattctgctcttggtcatgtgaacgtaacgataaaggagctcaggcgcctcttcttagttgatgatttagttgattctctgaagcaccaccatcaccatcactga

SEQ ID NO: 16 - protein, 599 aa, Artificial
  Fusion of Homo sapiens and non-Homo sapiens sequences
MKFLVNVALVFMVVYISYIYATSRSSGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGNDDDDKLSRDKRIYKGVIQAIQKSDEGHPFRAYLESEVAISEELVQKYSNSALGHVNVTIKELRRLFLVDDLVDSLKHHHHHH

SEQ ID NO: 17 - DNA, 43 bp, Artificial
  Synthetic primer
ctgctcttggtcatgtgaacgtaacgataaaggagctcaggcg

SEQ ID NO: 18 - DNA, 43 bp, Artificial
  Synthetic primer
cgcctgagctcctttatcgttacgttcacatgaccaagagcag